SEQUENCE LISTING (11 sequences)

SEQ ID NO: 1 - protein, 19 aa, Homo sapiens
  MOD_RES (10): PHOSPHORYLATION
TPVQNKRRRSVTPPEEQQE

SEQ ID NO: 2 - protein, 14 aa, Homo sapiens
  MOD_RES (7): PHOSPHORYLATION
QNKRRRSVTPPEEQ

SEQ ID NO: 3 - protein, 566 aa, Homo sapiens
  MOD_RES (339): PHOSPHORYLATION
MEVPQPEPAPGSALSPAGVCGGAQRPGHLPGLLLGSHGLLGSPVRAAASSPVTTLTQTMHDLAGLGSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 4 - protein, 539 aa, Homo sapiens
  MOD_RES (312): PHOSPHORYLATION
MEVPQPEPAPGSALSPAGVCGGAQRPGHLPGLLLGSHGLLGSPVRAAASSPVTTLTQTMHDLAGLGSETPKSQVGTLLFRSRSRLTHLSLSRRASESSLSSESSESSDAGLCMDSPSPMDPHMAEQTFEQAIQAASRIIRNEQFAIRRFQSMPDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 5 - protein, 580 aa, Homo sapiens
  MOD_RES (353): PHOSPHORYLATION
MEVPQPEPAPGSALSPAGVCGGAQRPGHLPGLLLGSHGLLGSPVRAAASSPVTTLTQTMHDLAGLGSETPKSQVGTLLFRSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 6 - protein, 601 aa, Homo sapiens
  MOD_RES (374): PHOSPHORYLATION
MEVPQPEPAPGSALSPAGVCGGAQRPGHLPGLLLGSHGLLGSPVRAAASSPVTTLTQTMHDLAGLGSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENVRFWKAGVGALREEEGACWGGSLACEDPPLPSWLQDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 7 - protein, 588 aa, Homo sapiens
  MOD_RES (361): PHOSPHORYLATION
MEVPQPEPAPGSALSPAGVCGGAQRPGHLPGLLLGSHGLLGSPVRAAASSPVTTLTQTMHDLAGLGSETPKSQVGTLLFRSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENVRFWKAGVGALREEEGACWGGSLACEDPPLPSWLQDGFVFKMPWKPTHPSSTHALAEWASRREAFAQRPSSAPDLMCLSPDRKMEVEELSPLALGRFSLTPAEGDTEEDDGFVDILESDLKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 8 - protein, 15 aa, Homo sapiens
  MOD_RES (1): PHOSPHORYLATION
SVTPPEEQQEAEEPK

SEQ ID NO: 9 - protein, 14 aa, Homo sapiens
QNKRRRSVTPPEEQ

SEQ ID NO: 10 - protein, 13 aa, Homo sapiens
  MOD_RES (7): PHOSPHORYLATION
MEVEELSPLALGR

SEQ ID NO: 11 - protein, 15 aa, Homo sapiens
SVTPPEEQQEAEEPK

(19) United States
(12) Patent Application Publication
(10) Pub. No.: US 2008/0008712 A1
(43) Pub. Date: Jan. 10, 2008

(54) Novel Phosphorylated Sequences of the Phosphatase Cdc25B, Antibodies for Said Sequences and Use Thereof

(75) Inventors: Bernard Ducommun, Belberaud (FR); Bernard Monsarrat, Albi (FR); Claude Prigent, Thorigne-Fouillard (FR)

Correspondence Address: YOUNG & THOMPSON, 745 SOUTH 23RD STREET, 2ND FLOOR, ARLINGTON, VA, 22202, US

(73) Assignees: CENTRE NATIONAL DE LA RECHERCHE SCIENTIFIQUE, F-75794 PARIS CEDEX 16 (FR); UNIVERSITE DE RENNES 1, F-35065 RENNES (FR); UNIVERSITE PAUL SABATIER TOULOUSE III, F-31062 TOULOUSE CEDEX 4 (FR)

(21) Appl. No.: 10/560,237

(22) PCT Filed: Jun. 8, 2004
(86) PCT No.: PCT/FR04/01416

§ 371 Date: May 15, 2006

(30) Foreign Application Priority Data
Jun. 12, 2003 (FR) 0307095

(51) Int. Cl. A61K 39/395; A61K 38/10; A61P 35/00; C07K 16/44; C12P 21/08; G01N 33/566; C07K 7/08; C07K 14/00; A61K 38/16

(52) U.S. Cl. 424/141.1; 435/69.1; 436/501; 514/13; 530/326; 530/327; 530/352; 530/387.9

(57) ABSTRACT

The present invention relates to a peptide sequence characterized in that it comprises or is constituted by a fragment of at least approximately 10 amino acids originating from the following sequence SEQ ID NO: 1:

| | |
|---|---|
| TPVQNKRRRSₚVTPPEEQQE | SEQ ID NO: 1 |

in which the serine residue in position 10 is phosphorylated,
said above-mentioned fragment containing said phosphorylated serine residue.

The present invention also relates to a polyclonal or monoclonal antibody capable of recognizing a peptide sequence as defined above.

A subject of the present invention is novel phosphorylated sequences of CDC25B phosphatase as well as polyclonal or monoclonal antibodies directed against these sequences. A subject of the present invention is also the use of these novel phosphorylated sequences in particular for implementing a method for in vitro diagnosis of cancers in humans or animals.

The mechanisms which control the division of cells bring into play numerous actors the activities of which are regulated by phosphorylation and dephosphorylation reactions, involving kinases and phosphatases. Deregulations of these mechanisms have been identified in numerous cancers. Their identification and their characterization today offer new prospects for the diagnosis and treatment of cancerous diseases.

CDC25B is a phosphatase which regulates the cell cycle and is essential for the control of the entry into mitosis. It belongs to a family which comprises three members coded by different genes (CDC25A, B and C) in mammals. The CDC25B protein is expressed and active at the end of the G2 phase of the cell cycle (Baldin et al., 1997; Gabrielli et al., 1996). Its intracellular location is regulated by NES and NLS sequences (Davezac et al., 2000) and by its interaction with the 14-3-3 proteins (Mils et al., 2000; Forrest et al., 2001). It has been suggested that CDC25B can act as a “starter” for early mitotic events (Nilsson et al., 2000). It could play a role in the initial activation of a CDC2/cyclin B population at the level of the centrosome before its nuclear translocation (Kumagai et al., 1992; Hoffmann et al., 1993). CDC25B activates the CDK/cyclin complexes to allow the architectural and biochemical rearrangements which are necessary in order to make the cell division process possible. Its activity is regulated by the variations in its expression, by its association with regulating partners and by phosphorylation events.

The Aurora A protein kinase, also known by the name STK5, is overexpressed in many breast tumors. This expression is correlated with a high tumor grade (Bischoff et al., 1998; Zhou et al., 1998). This kinase is coded by the STK15 gene located at the locus 20q13, an amplicon present in many tumors. This protein is located at the level of the centrosome (Dutertre et al., 2002). Its function appears to be important for the separation of the centrosomes (Giet et al., 2000), their duplication (Zhou et al., 1998) and the assembly of a bipolar mitotic spindle (Giet et al., 2000). The inhibition of its function by the RNA interference technology leads to the formation of monopolar spindles and its overexpression is responsible for a centrosomal amplification and a polyploidization (Meraldi et al., 2002; Bischoffet al., 1998; Zhou et al., 1998).

Currently, the identification of the substrates of Aurora A kinase is still very fragmented. CDC25B phosphatase is the first substrate identified which is also colocalized at the level of the centrosomes and plays a clear role in the control of the cell cycle and proliferation.

The present invention results from the inventors demonstrating the in vitro phosphorylation site of CDC25B phosphatase by Aurora A kinase, and the identification of the phosphorylated sequence of the splicing variant CDC25B3 of CDC25B on the serine residue in position 353.

One of the aims of the present invention is to provide novel phosphorylated sequences of the different variants of CDC25B phosphatase.

Another aim of the invention is to provide a novel antibody directed against a phosphorylated CDC25B phosphatase, said antibody being able to be used in the context of a medical diagnosis, or for the preparation of screens for the identification of molecules binding said phosphorylated sequence and which can represent novel agents which can be used in antitumoral pharmacology.

Another aim of the invention is to provide a novel tool for the study of molecular mechanisms which lead to polyploidization and to cell transformation, as well as a novel tool allowing the activity of the Aurora A kinase to be demonstrated on one of its physiological substrates, and consequently the identification of any quantitative temporal and spatial potential interference of this activity.

The present invention relates to a peptide sequence characterized in that it comprises or is constituted by a fragment of at least approximately 10 amino acids originating from the following sequence SEQ ID NO: 1:

| | |
|---|---|
| TPVQNKRRRSₚVTPPEEQQE | SEQ ID NO: 1 |

in which the serine residue in position 10 is phosphorylated, in particular by in vitro treatment of the sequence SEQ ID NO: 1 by the Aurora A kinase,

said above-mentioned fragment containing said phosphorylated serine residue.

The expression “phosphorylated residue” designates an amino acid carrying a phosphate group.

The present invention also relates to a peptide sequence as defined above, characterized in that it comprises or is constituted by the following sequence SEQ ID NO: 2:

| | |
|---|---|
| QNKRRRSₚVTPPEEQ | SEQ ID NO: 2 |

in which the serine residue in position 7 is phosphorylated.

The sequence SEQ ID NO: 2 corresponds to a fragment of the above-mentioned sequence SEQ ID NO: 1. More precisely, it corresponds to the fragment of SEQ ID NO: 1 delimited from the amino acid in position 4 to the amino acid in position 17.

The present invention also relates to a peptide sequence as defined above, characterized in that it comprises or is constituted by one of the following sequences:
sequence SEQ ID NO: 3, representing the splicing variant CDC25B1 of the protein of human origin of CDC25B phosphatase, the serine residue in position 339 of which is phosphorylated,
sequence SEQ ID NO: 4, representing a splicing variant CDC25B2 of the protein of human origin of CDC25B phosphatase, the serine residue in position 312 of which is phosphorylated,
sequence SEQ ID NO: 5, representing a splicing variant CDC25B3 of the protein of human origin of CDC25B phosphatase, the serine residue in position 353 of which is phosphorylated,
sequence SEQ ID NO: 6, representing a splicing variant CDC25B4 of the protein of human origin of CDC25B phosphatase, the serine residue in position 374 of which is phosphorylated,
sequence SEQ ID NO: 7, representing a splicing variant CDC25B5 of the protein of human origin of CDC25B phosphatase, the serine residue in position 361 of which is phosphorylated.

The present invention also relates to a polyclonal or monoclonal antibody capable of recognizing a peptide sequence as defined previously.

An advantageous polyclonal antibody of the invention is characterized in that it is capable of recognizing the sequence SEQ ID NO: 2 as defined above.

Such an antibody directed against the phosphorylated epitope of sequence SEQ ID NO: 2 is generated by immunizing rabbits with said epitope.

More precisely, said epitope is coupled in a covalent manner with a carrier protein such as hemocyanin, BSA or ovalbumin. The rabbits are then immunized over 3 months (4 injections in total) and the final bleeding allows the recovery of approximately 50 ml of serum. The serum is then double-purified by affinity on a phosphorylated peptide column then on a non-phosphorylated peptide column.

The present invention also relates to a method for the preparation of a monoclonal antibody as defined above directed against the peptide sequence SEQ ID NO: 2 as defined above, characterized in that it comprises the following steps:
the immunization of an animal by injection of the peptide sequence, SEQ ID NO: 2 as defined above,
the fusion between myelomas of an animal and splenocytes of an animal in order to obtain hybridomas,
the culturing of the hybridomas thus obtained,
the recovery and purification by cloning of a hybridoma, chosen from those obtained in the previous step and secreting an antibody directed against the peptide sequence SEQ ID NO: 2 as defined above.

The animal used for the immunization step is in particular a mouse.

The myelomas used for the fusion originate in particular from a mouse.

The splenocytes used for the fusion originate from an animal of the same species as that from which the myelomas originate, namely in particular a mouse.

The hybridomas which secrete the antibodies against the peptide sequence SEQ ID NO: 2 are chosen on the basis of the production of antibodies capable of recognizing, in an ELISA test, the phosphorylated peptide used for immunization but not the non-phosphorylated peptide.

The present invention also relates to a pharmaceutical composition characterized in that it contains, as active ingredient, a peptide sequence as defined previously, an antibody as defined above, or an anti-idiotypic antibody as defined above, in combination with a pharmaceutically acceptable vector.

An advantageous pharmaceutical composition according to the invention is characterized in that it contains, as active ingredient, the peptide sequence represented by the sequence SEQ ID NO: 2.

The present invention also relates to the use of a peptide sequence as defined above, an antibody as defined above, or an anti-idiotypic antibody such as defined above, for the preparation of medicaments intended for the treatment of cancers, such as breast cancers.

The present invention also relates to the use of an antibody as defined above, for implementing a method for in vitro diagnosis of cancers in humans or animals, in particular breast cancers.

According to an advantageous embodiment, the present invention relates to the use of a polyclonal antibody as defined above directed against the phosphorylated epitope of sequence SEQ ID NO: 2, for implementing a method for in vitro diagnosis of cancers in humans or animals, in particular breast cancers.

The present invention also relates to a method for in vitro diagnosis of cancers, in particular breast cancers, in humans or animals, characterized in that it comprises:
placing an antibody as defined above in the presence of a biological sample taken from an individual, said antibody if appropriate being fixed on a solid support,
the detection of a peptide sequence as defined above, which can be present in the biological sample using labelled reagents, in particular labelled antibodies, recognizing either the antibody bound to said peptide sequence, or the peptide sequence bound to said antibody in the complexes formed during the previous step between the antibody and the peptide sequence which can be present in the biological sample, this occurring, if appropriate, after suitable rinsing of the solid support.

The present invention also relates to a method of in vitro prognosis for cancers, in particular breast cancers, in humans or animals, characterized in that it comprises:
placing an antibody such as defined above in the presence of a tumor sample taken from an individual, said antibody if appropriate being fixed on a solid support,
the detection of a peptide sequence as defined above, which can be present in the biological sample, using labelled reagents, in particular labelled antibodies, recognizing either the antibody bound to said peptide sequence, or the peptide sequence bound to said antibody in the complexes formed during the previous step between the antibody and the peptide sequence which can be present in the biological sample, this occurring, if appropriate, after suitable rinsing of the solid support.

The present invention also relates to the use of the above-mentioned antibodies of the invention directed against a phosphorylated sequence of CDC25B in the context of implementing a diagnostic test the aim of which is to detect in tumoral samples the presence or not of this phosphorylated sequence, for the purpose of a diagnosis or a prognosis.

The present invention also relates to a method for screening a molecule capable of binding to a peptide sequence as defined above, said molecule being able to be used as an antitumoral agent or antiproliferative agent both in cells in culture and in a living organism or also against infectious agents (parasites, pathogenic fungi), characterized in that it comprises:
placing said molecule in the presence of the above-mentioned peptide sequence, and
the detection of the binding of said molecule by the use of appropriate competition methods, in particular by competition with the bond of an antibody as defined above.

The bond between said molecule and the phosphorylated peptide sequence can be detected according to the following method: the phosphorylated sequence (phosphorylated substrate) is bound to a solid support; the incubation with the above-mentioned antibody in solution then allows its fixation which is revealed by the use of a secondary antibody carrying a chromophore or by the direct labelling of the primary antibody (antibody of the invention directed against the phosphorylated sequence). The simultaneous incubation with a compound capable of binding said phosphorylated sequence leads to its fixation and the masking of the site recognized by the antibody. Visualization of this interaction can therefore be carried out and quantified by the reduction in the binding of the antibody.

### DESCRIPTION OF THE FIGURES

FIG. 1 represents a mass spectrum of the monophosphorylated peptide, 353-S₍ₚ₎VTPPEEQQEAEEPK-367. The abscissa axis corresponds to the ratio m/z and the ordinate axis corresponds to the percentage of relative abundance.

FIGS. 2A, 2B and 2C represent the results of western blot analyses with the monoclonal antibody SE96 (FIG. 2A), with the anti-αMBP antibody (New England Biolabs) (FIG. 2B) and with the anti-αAurora A antibody (see French Patent Application 02/07212) (FIG. 2C). In these figures, the first lane corresponds to the Aurora A protein kinase; the second lane to a recombinant protein MBP-CDC25B; the third lane corresponds to the protein kinase Aurora A and to the recombinant protein MBP-CDC25B and the fourth lane corresponds to MBP alone.

FIGS. 3a to 3h represent indirect immunofluorescence images produced in HeLa cells with the SE96 antibody.

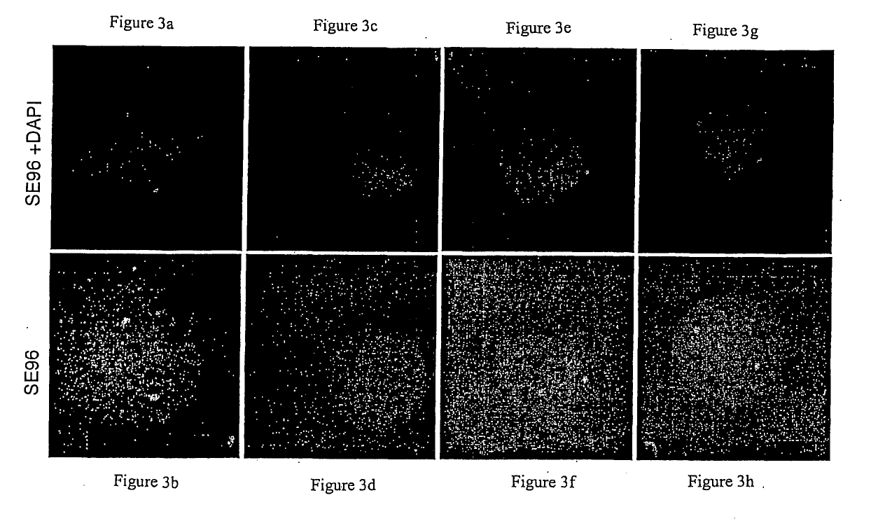

In FIGS. 3a, 3c, 3e and 3g, the HeLa cells have been fixed and used to carry out an immunofluorescence analysis with the SE96 antibodies and they have also been stained with DAPI.

In FIGS. 3b, 3d, 3f and 3h, the HeLa cells have been fixed and used to carry out an immunofluorescence analysis with the SE96 antibodies.

The HeLa cells of FIGS. 3c and 3d were put in competition with the phosphorylated peptide which served for the immunization (SEQ ID NO: 2); the HeLa cells of FIGS. 3e and 3f were put in competition with the non-phosphorylated peptide (QNKRRRSVTPPEEQ); and the HeLa cells of FIGS. 3g and 3h were put in competition with a phosphorylated peptide with no relation to the serine 353 (MEVEELS₍ₚ₎PLALGR).

These figures demonstrate that the labelling observed with the SE96 antibody is indeed eliminated by the immunogenic peptide in its phosphorylated form, but not by the same non-phosphorylated peptide. Moreover, an irrelevant phosphorylated peptide has no competitive effect, demonstrating the specificity vis-à-vis the phosphorylated sequence and not the presence of the phosphate group only.

### METHODS AND RESULTS

The Recombinant Aurora A Kinase Phosphorylates CDC25B3 on the Serine 353

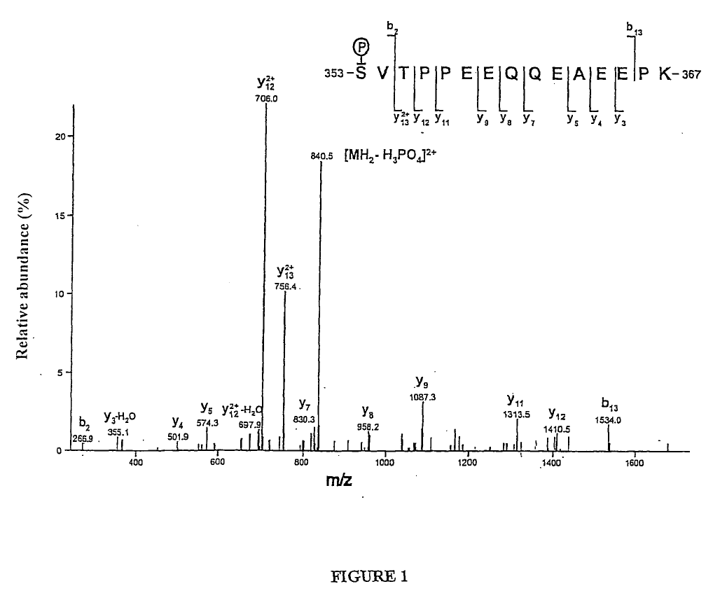

The CDC25B3 recombinant protein is phosphorylated in vitro by the recombinant Aurora A kinase. The product of the phosphorylation reaction was analyzed by mass spectrometry after excision of the electrophoresis gel and triptych digestion. The MS/MS spectrum of the monophosphorylated peptide, 353-SVTPPEEQQEAEEPK-367 is shown in FIG. 1. Its analysis indicates that it is the serine 353 which is phosphorylated by the kinase.

Similarly, it has been shown that the recombinant Aurora A kinase phosphorylates CDC25B1 on the serine 339, CDC25B2 on the serine 312, CDC25B4 on the serine 374 and CDC25B5 on the serine 361.

Production of Antibodies Against the CDC25B Protein Phosphorylated by the Aurora A Kinase

The peptide of sequence QNKRRRS(p)VTPPEEQ (SEQ ID NO: 2) was used for the immunization of rabbits. After sacrificing the animals, the serum was purified by chromatography in two steps: the first on a phosphorylated peptide column in order to retain the specific antibodies, then the second on a column of the same non-phosphorylated peptide of sequence QNKRRRSVTPPEEQ, so as to purify, in the eluate, the specific antibodies of the phosphorylated form. The recognition of the phosphorylated peptide by the antibodies was validated in an ELISA test. In the remainder of the document, these antibodies are designated by the name SE96.

The SE96 Antibody Recognizes CDC25B Phosphorylated by Aurora A

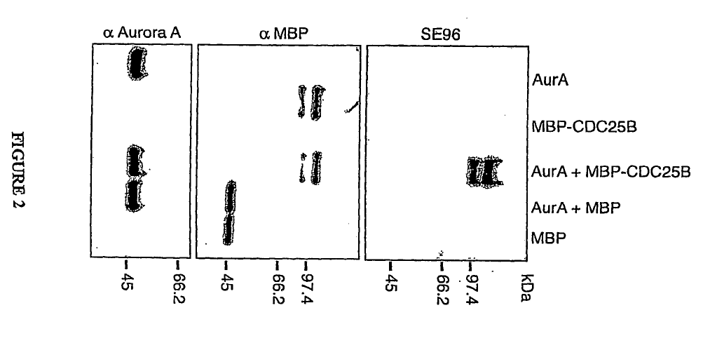

Recombinant proteins CDC25B-MBP (Maltose Binding protein) or MBP alone were incubated in the presence or not of Aurora A kinase. The samples were then analyzed by protein transfer (western blot) with the SE96 antibody and antibodies allowing the recognition of MBP and Aurora A. As shown in FIG. 2, the CDC25B protein phosphorylated by Aurora A is recognized by SE96, which validates the use of this antibody in a Western blot test.

The CDC25B Protein Phosphorylated on the Serine 353 is Located at the Level of the Centrosome

HeLa cells were fixed and used to carry out an immunofluorescence analysis with the SE96 antibodies. The cells were also stained with 4′-6 diamino-2-phenylindole (DAPI) in order to locate the nucleus. The images shown in FIG. 3 are representative of observations of a large number of cells. They show that the CDC25B protein phosphorylated on the serine 353 is located at the level of the centrosomes of the cells undergoing mitosis. This labelling is abolished when there is competition with the phosphorylated peptide which served for the immunization (SEQ ID NO: 2), but not with the non-phosphorylated peptide (QNKRRRSVTPPEEQ) or with a phosphorylated peptide with no relation to the serine 353 (MEVEELS(p)PLALGR). These observations validate the use of this reagent in immunofluorescence.

### REFERENCES

Baldin, V., Cans, C., Superti-Furga, G. & Ducommun, B (1997) Alternative splicing of the human CDC25B tyrosine phoshatase. Possible implications for growth control? Oncogene, 14, 2485-2495,
Bischoff, J. R. et al. (1998) A homologue of Drosophila aurora kinase is oncogenic and amplified in human colorectal cancers, EMBO J, 17, 3052-65,
Davezac, N; et al. (2000) Regulation of CDC25B phosphatases subcellular localization, Oncogene, 19, 2179-85,
Dutertre, S., Descamps, S. & Prigent, C. (2002) On the role of aurora-A in centrosome function, Oncogene, 21, 6175-83,
Forrest, A. & Gabrielli, B. (2001) Cdc25B activity is regulated by 14-3-3, Oncogene, 20, 4393-401,
Gabrielli, B. G. et al. (1996) Cytoplasmic accumulation of CDC25B phosphatase in mitosis triggers centrosomal microtubule nucleation in HeLa cells, J. Cell. Science, 109, 1081-1093,
Giet, R. & Prigent, C. (2000) The Xenopus laevis aurora/Ip11p-related kinase pEg2 participates in the stability of the bipolar mitotic spindle, Exp Cell Res, 258, 145-51,
Giet, R. et al. (2002) Drosophila Aurora A kinase is required to localize D-TACC to centrosomes and to regulate astral microtubules, J Cell Biol, 156, 437-51,
Hoffmann, I., Clarke, P., Marcote, M. J., Karsenti, E. & Draetta, G. (1993) Phosphorylation and activation of human cdc25-C by cdc2-cyclin B and its involvement in the self amplification of MPF at mitosis, EMBO J, 12, 53-63,
Kumagai, A. & Dunphy, W. (1992) Regulation of the cdc25 protein during the cell cycle in Xenopus extracts, Cell, 70, 139-151,
Meraldi, P., Honda, R. & Nigg, E. A. (2002) Aurora-A overexpression reveals tetraploidization as a major route to centrosome amplification in p53−/− cells, Embo J, 21, 483-92,
Mils, V. et al. (2000) Specific interaction between 14.3.3 isoforms and the human CDC25B phosphatase, Oncogene, 19, 1257-1265,
Nilsson, I. & Hoffmann, I. (2000) Cell cycle regulation by the Cdc25 phosphatase family, Prog Cell Cycle Res, 4, 107-14,
Zhou, H. et al. (1998) Tumor amplified kinase STK15/BTAK induces centrosome amplification, aneuploidy and conversion, Nat Genet, 20, 189-93.

1-11. (canceled)

12. Peptide sequence characterized in that it comprises or is constituted by a fragment of at least approximately 10 amino acids originating from the following sequence SEQ ID NO: 1:

| | |
|---|---|
| TPVQNKRRRSₚVTPPEEQQE | SEQ ID NO: 1 |

in which the serine residue in position 10 is phosphorylated,
said above-mentioned fragment containing said phosphorylated serine residue.

13. Peptide sequence according to claim 12, characterized in that it comprises or is constituted by the following sequence SEQ ID NO: 2:

| | |
|---|---|
| QNKRRRSₚVTPPEEQ | SEQ ID NO: 2 |

in which the serine residue in position 7 is phosphorylated.

14. Peptide sequence according to claim 12, characterized in that it comprises or is constituted by one of the following sequences:
sequence SEQ ID NO: 3, representing the CDC25B1 splicing variant of the protein of human origin of CDC25B phosphatase, the serine residue in position 339 of which is phosphorylated, or
sequence SEQ ID NO: 4, representing a CDC25B2 splicing variant of the protein of human origin of CDC25B phosphatase, the serine residue in position 312 of which is phosphorylated, or
sequence SEQ ID NO: 5, representing a CDC25B3 splicing variant of the protein of human origin of CDC25B phosphatase, the serine residue in position 353 of which is phosphorylated, or
sequence SEQ ID NO: 6, representing a CDC25B4 splicing variant of the protein of human origin of CDC25B phosphatase, the serine residue in position 374 of which is phosphorylated, or
sequence SEQ ID NO: 7, representing a CDC25B5 splicing variant of the protein of human origin of CDC25B phosphatase, the serine residue in position 361 of which is phosphorylated.

15. Polyclonal or monoclonal antibody capable of recognizing a peptide sequence according to claim 12.

16. Polyclonal antibody capable of recognizing the sequence SEQ ID NO: 2 as defined in claim 13.

17. Process for the preparation of a monoclonal antibody directed against the peptide sequence SEQ ID NO: 2 as defined in claim 13, characterized in that it comprises the following steps:
the immunization of an animal by injection of the peptide sequence according to claim 13,
the fusion between myelomas of an animal and splenocytes of an animal in order to obtain hybridomas,
the culturing of the hybridomas thus obtained, and
the recovery and purification by cloning of a hybridoma, chosen from those obtained in the previous step and secreting, an antibody directed against the peptide sequence according to claim 13.

18. Pharmaceutical composition characterized in that it contains, as active ingredient, a peptide sequence according to claim 12, a polyclonal or monoclonal antibody capable of recognizing said peptide sequence in association with a pharmaceutically acceptable vector.

19. A method for the treatment of cancers, such as breast cancers, comprising the administration of an appropriate amount of a peptide sequence according to claim 12 and a polyclonal or monoclonal antibody capable of recognizing said peptide sequence to a patient in need thereof.

20. A method for in vitro diagnosis or prognosis of cancers in humans or animals, in particular breast cancers, comprising the administration of an antibody according to claim 15 to a patient in need thereof.

21. Method for in vitro diagnosis or prognosis of cancers, in particular breast cancers, in humans or animals, characterized in that it comprises:
placing a polyclonal or monoclonal antibody capable of recognizing a peptide sequence according to claim 12 in the presence of a biological sample taken from an individual, said antibody being, if appropriate, fixed on a solid support, and
the detection of said peptide sequence according to claim 12, which is liable to be present in the biological sample using labelled reagents, in particular labelled antibodies, recognizing either the antibody bound to said peptide sequence, or the peptide sequence bound to said antibody in the complexes formed during the previous step between the antibody and the peptide sequence which is liable to be present in the biological sample, this occurring, if appropriate, after suitable rinsing of the solid support.

22. Method for screening a molecule capable of binding to a peptide sequence according to claim 12, said molecule being liable to be used as an antitumoral agent or antiproliferative agent, characterized in that it comprises:
placing said molecule in the presence of the above-mentioned peptide sequence, and
the detection of the binding of said molecule by the use of appropriate competition methods, in particular competition with the binding of a polyclonal or monoclonal antibody capable of recognizing said peptide sequence.
